(19) United States
(12) Patent Application Publication
(10) Pub. No.: US 2003/0208166 A1
(43) Pub. Date: Nov. 6, 2003

(54) Implantable device with free-flowing exit and uses thereof

(76) Inventor: Anthony H. Schwartz, Owings Mills, MD (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/427,512

(22) Filed: May 1, 2003

Related U.S. Application Data
(60) Provisional application No. 60/380,126, filed on May 6, 2002.

(51) Int. Cl. A61M 25/00

(52) U.S. Cl. 604/266; 604/269; 604/508; 427/2.1

(57) ABSTRACT

The present invention provides means for keeping the lumens and the distal orifices of catheters and like articles free of undesirable granulation tissues, granulomas, and the like. The catheter of the invention overcomes the problem of catheter occlusion by providing an immediately available source of an anti-neoplastic agent that minimizes or prevents formation of granulation tissue or granulomas at the tip and/or in the lumen of the catheter, thereby maintaining catheter patency. The catheter comprises a body with an exterior surface and a distal end, wherein at least the distal end comprises an anti-neoplastic agent to prevent or minimize catheter occlusion when the distal end of the catheter is in contact with a bodily tissue and/or fluid. In one embodiment, the anti-neoplastic agent is applied to the catheter as a coating. In another embodiment, the anti-neoplastic agent is an integral part of the catheter material.

### CROSS-REFERENCE TO RELATED APPLICATIONS

This application claims priority to U.S. Provisional Application Serial No. 60/380,126, filed May 6, 2002.

### BACKGROUND OF THE INVENTION

1. Field of the Invention

This invention relates to implantable catheters and like devices which are coated or compounded with an anti-neoplastic agent which prevents fibroblast proliferation or formation of granulation tissue or granulomas, thereby maintaining catheter patency.

2. Description of the Prior Art

Catheters have long found innumerable applications in a wide variety of medical procedures, including both therapeutic and diagnostic procedures. Catheters are useful, for example, as passageways for delivery of fluids to a patient and removal of fluids from the patient. They are thus routinely employed to conduct fluids containing medicaments from a source thereof directly to the tissue of an internal organ. Generally, a catheter is a hollow, tubular cannula that is capable of being inserted into canals, vessels, passageways, or other body cavities so as to permit injection or withdrawal of fluids, or to keep a passage open. Other catheter devices are used for controlling, directing and placing medical devices, such as intubation tubes or dilation catheters, into a body cavity, such as the trachea, a blood vessel, or the heart. These types of insertion catheters are commonly referred to as intubators, insertion sheaths, and/or dilators. Given that catheters are used for such a wide variety of applications, catheters are implemented in a variety of designs, shapes and sizes. However, when used, almost all catheters share the universal characteristic of having to be passed through the skin and subcutaneous tissue of the patient so as to be inserted into the proper body cavity

In many patient treatment applications, it is necessary or desirable for the catheter to remain in place for an extended period of time, which may range from several days to several years. For example, in recent years implantable catheters have been developed for use as access ports for insulin therapy, chronic pain management, chemotherapy, nutrition, and peritoneal dialysis. Other applications may include delivery of medicaments such as hypertensive medications, other cardiovascular medications, and medications to treat disorders of the brain and endocrine system. However, the implanted catheter lumen is susceptible to a number of complications such as occlusion. In some forms, catheter occlusions comprise granulation tissue or granulomas. When an occlusion occurs, the catheter must be replaced or the lumen otherwise cleared before infusion of the medical fluids can be resumed. However, occlusion removal in an implanted catheter can be difficult, and removal is not a desirable alternative.

Catheter occlusion can occur as a result of complex interactions involving the catheter material and the simultaneous presence of infusion and body fluids. It is known that the human body spontaneously rejects or encapsulates a foreign body such as a catheter that has been introduced into the body or a specific bodily organ. In some cases, encapsulation will impede or halt infusion through blockage of the catheter. Essentially, the body's own natural defense systems frustrate the potentially beneficial procedure of directly removing or supplying fluid to the tissue.

The reaction of living tissue to an implant can take a number of different forms. For example, the initial response to the surgical trauma of implantation is usually called the acute inflammatory reaction and is characterized by an invasion of polymorphonuclear leukocytes (PMNs). The acute inflammatory reaction is followed by the chronic inflammatory reaction, which is characterized by the presence of numerous macrophages and lymphocytes, some monocytes and granulocytes. Fibroblasts also begin accumulating in the vicinity of the implant and begin producing a matrix of collagen. The macrophages attempt to phagocytize the implant. However, if the implant is too large to be engulfed by the macrophages and is of a material resistant to digestion by the macrophages, these macrophages fuse together to form multinucleate foreign body giant cells. Macrophages and giant cells are the most common type of cell around many types of implants. The fibroblasts and collagen form a connective tissue capsule around the implant and the chronic inflammatory cells to effectively isolate the implant and these cells from the rest of the body. Connective tissue consisting of a fine network of collagen with active producing fibroblasts accompanied by chronic inflammatory cells, capillaries and blood vessels is referred to collectively as granulation tissue.

Thus, when a device such as a catheter is implanted into a soft tissue bed of a living organism such as a human or an animal, a granulation tissue or granulomas are formed around the catheter orifice comprising inflammatory cells, immature fibroblasts and blood vessels. This tissue capsule usually increases in thickness with time and subsequently occludes the catheter.

In the past, several methods have been proposed in an effort to prevent catheter occlusions or otherwise to clear the catheter lumen after a blockage has occurred. For example, heparin is well known for its anticoagulant characteristics, and is frequently used to prevent clot formation within the catheter lumen. In one approach, the catheter lumen is simply dipped in a heparin solution before patient placement, with the dip coating being generally effective to prevent localized clotting over a relatively short period of time until the heparin is degraded upon contact with body fluids. Unfortunately, heparin is ineffective to dissolve clots and/or other occlusions after formation thereof. Moreover, heparin has not been approved for use with some medications, such as insulin.

Alternative occlusion control methods have utilized a fibrinolytic enzyme such as a kinase enzyme known to be effective in dissolving fibrin-based clots. However, in the presence of body fluids, the fibrinolytic enzyme degrades rapidly and is thus ineffective for long-term occlusion control. Any clots formed subsequent to enzyme degradation are extremely difficult to dissolve, since it is difficult to deliver additional enzyme solution to the blockage site along the catheter lumen. Others methods include loading or compounding the catheter with an anti-inflammatory agent.

There exists, therefore, a significant need for further improvements in implantable catheters and catheter assemblies to prevent or minimize catheter occlusions, particularly over an extended period of time.

### SUMMARY OF THE INVENTION

The invention relates to means for keeping the lumens and the distal orifices of catheters and like articles free of undesirable tissues such as granulation tissues, granulomas, and the like.

More specifically, in accordance with the invention, an improved indwelling catheter to be positioned within or adjacent to a bodily tissue or organ is provided. The improved catheter overcomes the problem of catheter occlusion by providing an immediately available source of an anti-neoplastic agent that minimizes or prevents formation of granulation tissue or granulomas at the tip and/or within the lumen of the catheter when the distal end of the catheter is in contact with a bodily tissue and/or fluid, thereby maintaining catheter patency. In one embodiment, the catheter comprises a body with exterior and interior surfaces and a distal end, wherein at least the distal end of the catheter surfaces are coated with an anti-neoplastic agent. In another embodiment, the anti-neoplastic agent is an integral part of the catheter material.

This invention further provides a drug delivery device for implantation into a mammal for drug delivery to a prescribed location. The device comprises an improved catheter of this invention that is adapted to prevent or minimize occlusion of the catheter orifice and lumen. In one embodiment of this device, the catheter is coupled at its distal end to a coil, wherein at least the distal end of the coil comprises an anti-neoplastic agent to prevent or minimize occlusion of the coil when the distal end of the coil is in contact with a bodily tissue and/or fluid.

The invention further provides a method of forming an improved catheter comprising the steps of providing a catheter; providing a composite formed of a polymeric material and an anti-neoplastic agent; and coating the exterior surface of at least the distal end of the catheter, whereby the anti-neoplastic agent will be present at the distal end of the catheter when the coated portion is in contact with the bodily tissue or organ.

The invention further provides a method of forming catheter loaded with an anti-neoplastic, said method comprising the steps of providing a composite solution of a polymer and an anti-neoplastic agent; and forming the catheter from the composite solution, whereby the anti-neoplastic agent will be present throughout the surface of the catheter when the catheter is in contact with bodily fluid.

Implantation sites for the catheter within a mammal include abdominal, subcutaneous, and peritoneal tissues, the brain, the neuraxis, the intrathecal space, the epidural space, peripheral nerves, sites that are in pain and respond to local anesthetic block, the intramedullary space, arteries, and other suitable organs or bodily tissues.

The improved catheters of this invention may be part of an implantable device that is implantable within a mammal and adapted for long-term efficient drug delivery to surrounding tissue. The device comprises improved catheters of this invention coupled to a drug delivery pump. The pump is optionally implanted under the skin and comprises a refillable medication reservoir from which medication is pumped or delivered by other means through the catheter to a predetermined site within a mammal. The improved catheter in these devices inhibits or minimizes ingrowth of tissues such as granulation tissues, granulomas, and the like at the tip and/or within the lumen of the catheter, while permitting the flow of drugs from the reservoir.

Examples of such devices include insulin pumps and devices for delivering chemotherapy or pain medication. Accordingly, another aspect of this invention provides a method of resetting the central nervous system of a patient suffering from pain, comprising:

(a) administering a continuous or intermittent dose of an analgesic or local anesthetic compound to the patient for a predetermined amount of time and at a rate sufficient to ameliorate pain, wherein said drug is administered with an improved catheter of this invention; and

(b) terminating said administration after the predetermined time; and

(c) observing whether said pain recurs in said patient after said termination.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURES

The accompanying drawings, which are incorporated in and form a part of the specification, illustrate non-limiting embodiments of the present invention, and together with the description serve to explain the principles of the invention.

In the Figures:

FIG. 1 is a sectional view illustrating the cross-sectional geometry of an indwelling portion of a catheter.

FIG. 2 is an enlarged, partial sectional view of one embodiment of a catheter of this invention coated with a material comprising an anti-neoplastic agent.

FIG. 3 is an enlarged, partial sectional view of an alternative embodiment of a catheter of this invention comprising an anti-neoplastic agent throughout the body of the catheter.

### DETAILED DESCRIPTION OF THE INVENTION

This invention relates generally to indwelling catheters, tubes, stents, and like articles that remain in a living body for extended periods. More particularly, this invention addresses problems that arise from accumulation of clogging deposits such as granulation tissue or granulomas on an indwelling catheter or like article at the distal orifice or within the lumen of such an article. Such deposits interfere mechanically and directly with the therapeutic function of a catheter or other tube, and may interfere with the therapeutic functions of various other indwelling articles. Such catheters, tubes and stents are used in medical procedures and therapies for both humans and animals. An “indwelling” catheter or like article is one that is implanted and left in place for protracted periods, such as fifteen minutes or longer.

As used herein, the term “catheter” refers to any tubular medical device for insertion into canals, vessels, passageways, cavities, tissues, or organs of a living mammal, usually to permit injection or withdrawal of fluids or to keep a passage open. The term “distal” is used to define the end of the catheter that is in contact with a bodily tissue or organ.

As noted above, a problem inherent in the implantation of a catheter or like device in a living mammal is fibroblastic proliferation and the encapsulation or occlusion of the catheter by granulation tissue or granulomas. As used herein, the term “occlusion” refers to an obstruction of a catheter lumen which results in diminished or blocked flow of fluid therethrough. The occlusion can occur at the orifice of the lumen and/or within the lumen. The catheters of this invention overcome this problem by providing an immediately available source of an anti-neoplastic agent that minimizes or prevents formation of granulation tissue or granulomas at the tip and/or in the lumen of the catheter, thereby maintaining catheter patency. More specifically, the catheters of this invention are either coated or compounded with the anti-neoplastic agent, wherein the anti-neoplastic agent is present at least at the distal end of the catheter. It is not necessary that the anti-neoplastic agent be released or leach from the catheter to be effective, and in certain applications it may be desirable that the anti-neoplastic remain within the coating or within the catheter material.

The term “granulation tissue” refers to a kind of tissue having a rough or irregular surface that is typically formed during would healing and comprises vascular connective tissue which contains new capillaries, fibroblasts, and inflammatory cells. A “granuloma” is an inflammatory swelling or growth composed of granulation tissue. In its most classical form, a granuloma comprises concentric layers of cells. At the center, there is often a focus of caseous necrosis, although this need not be the case. This central focus is surrounded by a layer of specialized macrophages (epithelioid cells) and multinucleated giant cells. The next layer is predominantly lymphocytes, and the outer layer is most often fibroblasts. Granulocytes, plasma cells, and other cell types may also be present. When formed as a result of a foreign body such as a catheter, the granuloma is of the non-immune type.

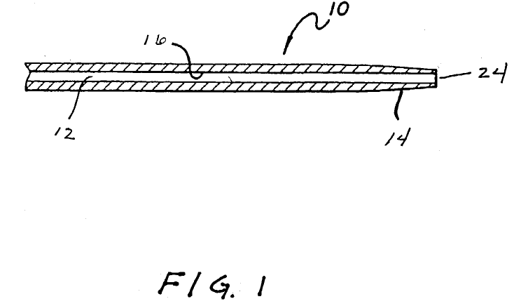

With reference now to FIG. 1, a catheter 10 has a generally conventional construction that includes an elongated tubular member defining a lumen 12. In one embodiment the catheter 10 is adapted for transcutaneous or subcutaneous placement in the patient for infusing medical fluids or medicaments to or drawing body fluids from the patient. Alternatively the catheter is designed to keep a passage such as a blood vessel opened. The catheter 10 may be installed with the assistance of an elongated stylet needle (not shown) or the like which can be withdrawn from the catheter lumen 12 subsequent to catheter placement. In yet another embodiment, the catheter may be a component of an implantable device adapted for use in combination with an implantable medication infusion pump or the like to continuously or intermittently deliver medication to a patient over an extended period of time.

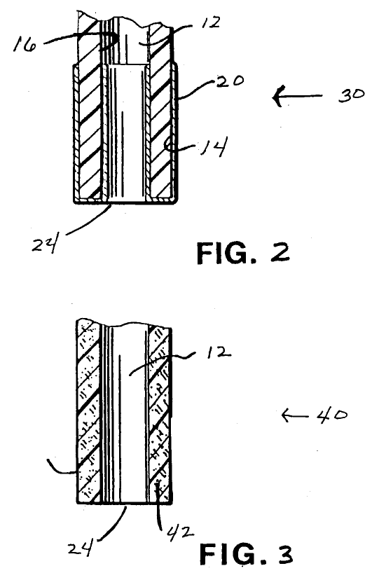

With reference to FIG. 2, in accordance with one embodiment of the present invention, a selected anti-neoplastic agent that prevents or minimizes formation of an occlusion is applied as an integral part of a coating on at least a portion (and preferably near the distal end) of both the exterior and interior surfaces of the catheter. In the embodiment depicted in FIG. 2, the exterior and interior surfaces 14 and 16, respectively, of at least the distal end 18 of the catheter 30 are provided with a coating 20 comprising a material loaded with an anti-neoplastic agent. The coating may be applied by a number of methods known in the art. For example, a composition is prepared by forming either a dissolved solution or a suspension of the anti-neoplastic agent in an appropriate coating material such as a polymer. The concentration of anti-neoplastic agent in the solution or suspension is an amount effective to prevent or minimize catheter occlusion in the final device. Either the distal end 18 or the entire catheter is then immersed into this composition. The composition coats the exterior and interior surfaces 14 and 16, respectively, of the catheter and, as it solidifies, encapsulates these surfaces in the polymer/anti-neoplastic agent formulation. The dried catheter thus includes a coating of the anti-neoplastic agent on its surfaces. Preferably, the immersion methods are adapted such that the solution or suspension does not completely fill the interior 12 of the catheter or block the orifice 24. Methods are known in the art to prevent such an occurrence, including adapting the surface tension of the solvent used to prepare the composition, clearing the lumen after immersion, and placement of an inner member with a diameter smaller than the lumen 12 in such a way that a passageway exists between all surfaces of the catheter and the inner member. By the use of the catheter shown in FIG. 2, an anti-neoplastic agent is present at the outer surface of at least the distal end 18 of the catheter when the catheter is in contact with bodily tissues, organs or fluids and acts to prevent or minimize occlusion of the catheter orifice 24 or lumen 12. An alternative to dipping the distal end 18 of the catheter is to spray-coat the exterior and interior surfaces 14 and 16, respectively, with a vaporized form of the composition comprising the anti-neoplastic agent.

In yet another embodiment (not shown), a thin layer of an anti-neoplastic agent is covalently bonded to the exterior and interior surfaces 14 and 16, respectively, of the catheter. In this embodiment, the catheter surfaces are prepared to molecularly receive the anti-neoplastic agent according to methods known in the art. A binding agent may be needed between the anti-neoplastic agent molecules and the polymer molecules on the surface.

The polymer coating comprises any biocompatible polymeric material capable of holding the pharmaceutical agent to be delivered. Suitable materials include, but are not limited to, polycarboxylic acids, cellulosic polymers, gelatin, polyvinylpyrrolidone, maleic anhydride polymers, polyamides, polyvinyl alcohols, polyethylene oxides, glycosaminoglycans, polysaccharides, polyesters, polyurethanes, silicons, polyorthoesters, polyanhydrides, polycarbonates, polypropylenes, polyatic acids, polyglycolic acids, polycaprolactones, polyhydroxybutyrate valerates, polyacrylamides, polyethers, and mixtures and copolymers thereof.

Generally, the amount of coating to be placed on the catheter will vary with the polymer, and may range from about 0.1 to 40 percent of the total weight of the catheter after coating. The polymer coatings may be applied in one or more coating steps depending on the amount of polymer to be applied. Typically, the amount of anti-neoplastic agent represents about 0.1-40 percent by weight of the coating.

FIG. 3 depicts an alternative embodiment of the catheter according to the invention. In this embodiment, the anti-neoplastic agent is provided throughout the body 42 of the catheter 40 by mixing and compounding an anti-neoplastic agent directly into the catheter polymer melt before forming the catheter. For example, the anti-neoplastic agent can be compounded into materials such as silicone rubber or urethane. The compounded material is then processed by conventional method such as extrusion, transfer molding or casting to form a tubular configuration. The catheter 40 resulting from this process benefits by having an anti-neoplastic agent dispersed throughout the entire catheter body 42. Thus, the anti-neoplastic agent is present at the outer surface of the catheter when the catheter is in contact with bodily tissues, organs or fluids and acts to prevent or minimize occlusion of the catheter orifice 24 or lumen 12.

The invention is applicable, for example, to such catheters including, but not limited to, tracheal, cardiac, central and other venous catheters, double channel catheters having one lumen for irrigation and one lumen for aspiration, and the Foley urinary-tract catheter or other urinary catheters. The invention is also suitable for use with tubes that irrigate or drain body cavities including, but not limited to, the pleural, peritoneal, subarachnoid, intrathecal, subdural and intracraneal cavities, or tubes that are used for special procedures such as evacuation of hematoma. This invention is further applicable to other implantable devices such as (without exclusion) stents, stent grafts, and stent covers. For example, stents are often placed in the urinary tract to help with kidney stone removal. Thus, an anti-neoplastic coated or compounded stent in accordance with this invention would allow the stent to remain place for longer periods of time, since occlusion of the stent would be minimized or prevented as a result of the anti-neoplastic coating. This would have the added benefit of minimizing replacement of the urinary tract catheter, which is often performed under general anesthetic in order to reduce pain from the procedure.

In another embodiment, the catheter comprises a coil at the distal end, wherein the coil is coated with an anti-neoplastic agent. In this embodiment, the exterior and/or interior surfaces of the catheter lumen are optionally coated with the anti-neoplastic agent. Such coil-tipped catheters are particularly useful for short-term indications.

Many known anti-neoplastic agents can be utilized according to this invention to inhibit or minimize catheter occlusion caused by fibroblast proliferation, granulation tissue formation, and/or granuloma formation. As used herein, the term “anti-neoplastic agent” also encompasses agents which may not traditionally be classified as an anti-neoplastic agent but performs the same function of inhibiting or minimizing fibroblast proliferation, granulation tissue formation, and granuloma formation.

Examples of anti-neoplastic agents suitable for purposes of this invention include, but are not limited to, antibiotics including anthracylines and immunosuppressive macrolides such as rapamycin; anti-mitotic agents including taxoids such as paclitaxel, vinca alkaloids, and epipodophyllotoxins; aklylating agents such as nitrogen mustards and nitrosoureas; anti-metabolites such as folic acid antagonists, pyrimidine analogs, and purine analogs; hormones such as androgens, anti-androgens, progestins, estrogens, anti-estrogens, aromatase inhibitors, and gonadotropin-releasing hormone analogs; and biological response inhibitors such as interlukins.

This invention further provides a fluid delivery system for delivery of medicaments to a patient. In one embodiment, the delivery system comprises a drug reservoir, a peristaltic pump to pump out the drug from the reservoir, and an improved catheter of this invention to transport the drug from the reservoir to a desired location within the patient's anatomy. In another embodiment, the delivery system comprises a combined fluid pump and reservoir. Such devices also typically include a battery to power the pump as well as an electronic module to control the flow rate of the pump. The proximal end of the catheter is secured to the pump reservoir, whereas the opposite, distal end is adapted to be positioned adjacent to or within the tissue or organ intended to receive the medicament. This invention contemplates the use of any suitable pump-reservoir system known in the art. The delivery system is surgically implanted in the patient and programmed to deliver prescribed amounts of medicament continuously, on demand, or at regularly scheduled intervals. The fluid delivery system can be modified by providing an external source of fluid, rather than the internal pump, or can be used with external means such as a pump for withdrawing fluid from the body through the catheter.

As discussed, the catheters of this invention may be used to deliver therapeutic agents to a tissue or organ of a mammal. Such agents may include any medication which would be desirably applied locally to a specific internal tissue site that can be reached by the catheter or like article. A notable advantage of the invention is that the use of an implantable drug delivery device avoids the need for replacement of clogged catheters in the subject, which procedure often causes pain and inconvenience to the patient and increases the risk of infection. In addition, use of an implanted device increases patient compliance with a prescribed therapeutic regimen and affords greater mobility and easier outpatient management.

One specific therapeutic agent that can be advantageously delivered by a catheter or device of this invention is a drug for treatment of chronic pain. Increasingly, chronic pain is being recognized as a major medical problem. Chronic pain is persistent pain which has long outlasted the onset of any known or suspected physical cause. It can occur after a known injury or disease or it can occur without any known physical cause whatsoever. Moreover, it can be accompanied by known tissue pathology, such as chronic inflammation that occurs in some types of arthritis, or it can occur long after the healing of the injured tissue which is suspected or known to be the cause of chronic pain. Chronic pain is a very general concept and there are several varieties of chronic pain related to the musculoskeletal system, visceral organs, skin, and nervous system. Neuropathic pain can occur as a form of chronic pain and can also occur under acute conditions such as those following surgery or accidental trauma. Neuropathic pain can be defined as pain that results from an abnormal functioning of the peripheral and/or central nervous system. A critical component of this abnormal functioning is an exaggerated response of pain-related nerve cells either in the peripheral or in the central nervous system. This exaggerated responsiveness is manifested behaviorally as increased sensitivity to pain, i.e., as hyperalgesia or allodynia, both of which can occur in chronic neuropathic and acute inflammatory pains. Neuropathic pain is thought to be a consequence of damage to peripheral nerves or to regions of the central nervous system. However, abnormal functioning of pain-related regions of the nervous system call also occur with chronic inflammatory conditions such as certain types of arthritis and metabolic disorders such as diabetes as well as with acute inflammatory conditions. Thus, many types of chronic pains that are related to inflammation, as well as acute pains that are related to inflammation, can be considered to be at least partly neuropathic pains. Unfortunately, there are a significant number of chronic pain patients (approximately 5-10 percent) who have inadequate pain relief, intolerable side effects, or morbidity associated with oral medications or repeated surgeries.

The improved catheters of this invention therefore are desirable for delivering drugs such as analgesics or local anesthetics as a therapy for allowing chronic pain patients to become pain free. It has been observed in the field of pain management that delivery of a pain-relieving drug over a period of time “breaks the cycle” by blocking peripheral pain input, thereby allowing the central nervous system to “reset” itself back to its normal state. Once this state is reached, the patient may be pain free without requiring further administration of the drug. By using an improved catheter of this invention, the analgesic or local anesthetic compound can be delivered over a period of time necessary to allow the patient to eventually become pain free, while avoiding the complication of catheter occlusion.

Accordingly, this invention further provides devices for and methods of pain management in a subject comprising the steps of implanting a drug delivery device at an implantation site in the body of a subject, where the drug delivery device comprises an improved catheter of this invention and further is capable of controlled drug release, and delivering an analgesic or local anesthetic from the device to a delivery site for entering into the predetermined site in the patient in an amount effective to alleviate pain in the subject. A primary object of this embodiment is to provide a method for management of pain wherein the patient is provided with a therapeutic amount of drug such as an analgesic or local anesthetic drug for a period of time sufficient to allow the central or peripheral nervous system to reset itself back to its normal state. Once the nervous system is reset, the patient may be able to discontinue treatment with pain control drugs. The term “pain management or treatment” is used herein to generally describe regression, suppression, or mitigation of pain so as to make the subject more comfortable as determined by subjective criteria, objective criteria, or both. In general, pain is assessed subjectively by patient report, with the health professional taking into consideration the patient's age, cultural background, environment, and other psychological background factors known to alter a person's subjective reaction to pain.

More specifically, one aspect of this invention provides 11. A process for achieving effective and satisfactory relief from pain in a patient either temporarily or permanently, comprising:

(a) administering a continuous or intermittent dose of an analgesic or local anesthetic drug to the patient for a predetermined amount of time and at a rate sufficient to ameliorate said pain, wherein said drug is delivered via a catheter having a body with an exterior and interior surface and a distal end, wherein the exterior and interior surfaces of at least the distal end are coated with a material comprising an anti-neoplastic agent to prevent or minimize catheter occlusion when the distal end of the catheter is in contact with a bodily tissue and/or bodily fluid; and

(b) terminating said administration after said predetermined time; and

(c) observing whether said pain recurs in said patient after said termination.

If the pain does not recur after the drug administration is terminated, the patient may be able to discontinue the drug therapy. Alternatively, if the pain does recur, the above-described method is repeated as necessary until the patient is pain-free without the aid of a pain-reliving drug. The amount of time necessary to reach this pain-free state will of course vary depending on the source of the pain, the patient's health, age, and other criteria. Generally, the drug is administered for at least one to two weeks, and in certain situations is administered for one to two months or longer, until the pain-relieving drug is not longer needed or is no longer effective in relieving pain.

As one non-limiting example, spinal administration (intrathecal or epidural) of centrally acting analgesic compounds via an improved catheter of this invention, such as a spinal catheter, will be therapeutically effective for the treatment of chronic pain while eliminating the problem of catheter occlusion. To reduce systemic side effects caused by relatively high dosage via systemic delivery, direct spinal delivery of the analgesic compound is preferred. In this way, drug is delivered in a concentrated manner and at low doses to its specific site of action on receptors in the neuraxis, minimizing systemic side effects.

The analgesic or local anesthetic drug is typically delivered in a pattern, generally a substantially regular pattern, over a pre-selected period of time (e.g., other than a period associated with, for example a bolus injection). “Patterned” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “therapeutically effective amount” is meant an amount of a therapeutic agent, or a rate of delivery of a therapeutic agent, effective to facilitate a desired therapeutic effect. The precise desired therapeutic effect (e.g., the degree of pain relief, and source of the pain relieved, etc.) will vary according to the condition to be treated, the formulation to be administered, and a variety of other factors that are appreciated by those of ordinary skill in the art. In general, the method of the invention involves the suppression or mitigation of pain in a subject suffering from pain that may be associated with any of a variety of identifiable or unidentifiable etiologies.

Pain amenable to alleviation includes, but is not necessarily limited to various types of acute or chronic pain, including neuropathic pain, cancer pain, inflammatory disease pain, nociceptive pain, postoperative pain, iatrogenic pain, complex regional pain syndrome, failed-back pain, soft tissue pain, joint pain, bone pain, central pain, injury pain, arthritic pain, hereditary disease, infectious disease, headache, causalgia, hyperesthesia, sympathetic dystrophy, phantom limb syndrome, and denervation. This invention is particularly useful in the treatment of pain of long duration or chronic pain.

Potential classes of pain-relieving drugs for treatment of chronic pain according to this invention include analgesics, typically opioids, which act on opioid pain receptors. Examples of such opioid analgesics are morphine, fentanyl, bipuvacaine, sufentanil, alfentanil, hydromorphone, meperidine, methadone, buprenorphine, DADL, butorphanol and related opioids. Other potential drug classes include analgesics that act on non-opioid pain receptors. One such group of analgesics that act on non-opioid pain receptors are alpha-2 adrenergic receptor agonists such as clonidine, tizanidine, ST-91, medetomidine, dexmedetomidine and related alpha-2 adrenergic agonists. Another group of analgesics are NMDA receptor antagonists such as dexmethorphan, Ifenprodil, MK-801 and related NMDA antagonists. Yet another group of analgesics are somatostatin analogs such as Octreotide, Sandostatin, Vapreotide, Lanreotide and related somatostatin analogs. Other analgesics may be used that act on non-opioid pain receptors such as ketorolac, super oxide dismutase, baclofen, calcitonin, serotonin, vasoactive intestinal polypeptide, bombesin, omega-conopeptides and related non-opioid analgesics. In addition, local anesthetics or tricyclic antidepressants may be administered according to this invention for treatment of chronic pain.

Other examples of therapeutic agents that can be administered via an improved catheter or device of this invention include anti-thrombogenic agents or other agents for suppressing stenosis or late restenosis, such as anti-platelet derived growth factor or any other inhibitor of cell growth at the stenosis site. Additional examples include carbamazepine, divalproex sodium, membrane stabilizers such as valproic acid and phenytoin, skeletal muscle relaxants and centrally acting pain modulators such as baclofen, GABA enhancers such as clonazepam and other benzodiazapenes, and sirolimus.

The therapeutic agent also may comprise a vasodilator to counteract vasospasm, for example an antispasmodic agent such as papaverine. The therapeutic agent may be a vasoactive agent such as a calcium antagonist, or an alpha or beta-adrenergic agonist or antagonist.

Additionally, the therapeutic agent in accordance with this invention may be an anti-neoplastic agent such as 5-fluorouracil or any known anti-neoplastic agent, preferably mixed with a controlled release carrier for the agent, for the application of a persistent, controlled release anti-neoplastic agent to a tumor site.

The therapeutic agent may be an antibiotic which may be applied by this invention, preferably in conjunction with a controlled release carrier for persistence, to an infected stent or any other source of localized infection within the body. Similarly, the therapeutic agent may comprise steroids for the purpose of suppressing inflammation or for other reasons in a localized tissue site.

The therapeutic agent may also constitute any desired mixture of individual pharmaceuticals or the like, for the application of combinations of active agents

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

I claim:

1. A catheter having a body with an exterior and interior surface and a distal end, wherein the exterior and interior surfaces of at least the distal end are coated with a material comprising an anti-neoplastic agent to prevent or minimize catheter occlusion when the distal end of the catheter is in contact with a bodily tissue and/or bodily fluid.

2. The catheter of claim 1, wherein said anti-neoplastic agent is selected from the group consisting of alkylating agents, anti-metabolites, hormones, antibiotics, mitotic inhibitors, and biological response modifiers.

3. The catheter of claim 1, wherein said anti-neoplastic agent is rapamycin.

4. The catheter of claim 1, wherein said anti-neoplastic agent is paclitaxel.

5. The catheter of claim 1, wherein said coating material is a polymeric material.

6. A catheter having a body and a distal end to be positioned within or adjacent to a bodily tissue or organ, wherein the body of the catheter is formed of a polymeric material comprising anti-neoplastic agent dispersed throughout the polymeric material.

7. A drug delivery device for implantation within the body of a mammal, comprising a drug supply reservoir, a pump, and a catheter having a body with an exterior and interior surface and a distal end, wherein at least a portion of the exterior and interior surfaces of at least the distal end are coated with a material comprising an anti-neoplastic agent to prevent or minimize catheter occlusion when the distal end of the catheter is in contact with a bodily tissue and/or fluid.

8. A drug delivery device for implantation within the body of a mammal, comprising a drug supply reservoir, a pump, and a catheter having a body and a distal end to be positioned adjacent to bodily tissue for the conduction of fluid thereto or therefrom, wherein the body of the catheter is formed of a polymeric material comprising anti-neoplastic agent or anti-scarring agent being dispersed throughout the polymeric material.

9. A method of preventing or minimizing occlusion of a medical device having a catheter to be inserted into bodily tissue and having an exterior and interior surface, comprising coating at least a portion of said exterior and interior surfaces with a coating material comprising an anti-neoplastic agent.

10. A method of preventing or minimizing occlusion of a medical device having a catheter to be inserted into bodily tissue, comprising providing a composition comprising a polymer material and an anti-neoplastic agent, and forming said catheter from said composition.

11. A process for achieving effective and satisfactory relief from pain in a patient either temporarily or permanently, comprising:
(a) administering a continuous or intermittent dose of a pain-relieving drug to the patient for a predetermined amount of time and at a rate sufficient to ameliorate said pain, wherein said drug is delivered via a catheter having a body with an exterior and interior surface and a distal end, wherein the exterior and interior surfaces of at least the distal end are coated with a material comprising an anti-neoplastic agent to prevent or minimize catheter occlusion when the distal end of the catheter is in contact with a bodily tissue and/or bodily fluid;
(b) terminating said administration after said predetermined time; and
(c) observing whether said pain recurs in said patient after said termination.

12. The method of claim 11, wherein said drug is administered for at least two weeks.

13. A process for achieving effective and satisfactory relief from pain in a patient either temporarily or permanently, comprising:
(a) administering a continuous or intermittent dose of an analgesic or local anesthetic drug to the patient for a predetermined amount of time and at a rate sufficient to ameliorate pain, said drug delivered via a catheter having a body and a distal end to be positioned within or adjacent to a bodily tissue or organ, wherein the body of the catheter is formed of a polymeric material comprising anti-neoplastic agent dispersed throughout the polymeric material;
(b) terminating said administration after said predetermined time; and
(c) observing whether said pain recurs in said patient after said termination.
